(19) United States
(12) Patent Application Publication
(10) Pub. No.: US 2015/0041532 A1
(43) Pub. Date: Feb. 12, 2015

(54) Longitudinal Personal Health Management System Using Mobile Data Capture

(71) Applicant: Peter A. Altman, Menlo Park, CA (US)

(72) Inventor: Peter A. Altman, Menlo Park, CA (US)

(21) Appl. No.: 14/521,289

(22) Filed: Oct. 22, 2014

Related U.S. Application Data
(63) Continuation of application No. 12/269,005, filed on Nov. 11, 2008, now abandoned.
(60) Provisional application No. 60/987,077, filed on Nov. 11, 2007.

(51) Int. Cl. H04L 29/06; A61B 5/00; G06F 17/30; A61B 5/11

(52) U.S. Cl. CPC H04L 65/403; A61B 5/1118; A61B 5/4866; G06F 17/30091
USPC 235/375

(57) ABSTRACT

A system and method for tracking longitudinal data for the maintenance and management of health of an individual. The method entails marking numerous groceries, drugs, beverages, etc., with labels that may be read by a scanner or reader embedded in a PDA or cell phone or similar small electronic device, and providing users with a suitable scanner or reader. Users scan the labels of the products they consume during the course of a day, and the system keeps track of calories, fat content, carbohydrate content, etc. of the food consumed, drug content of the drugs consumed, etc. The system can also be operated to track exercise levels and energy expenditure during the day. The system is operable to provide a comparative display of various databased information, such as calorie intake versus calorie expenditure during exercise.

This application is a continuation of U.S. application Ser. No. 12/269,005 filed Nov. 11, 2008, which claims priority to U.S. Provisional Application 60/987,077 filed Nov. 11, 2007.

### FIELD OF THE INVENTION

The inventions described below relate to personal digital assistants, internet, and software tools to enable individuals to obtain, manage, and utilize health data.

### BACKGROUND OF THE INVENTION

Management of diet, health, and fitness has drawn increasing amounts of attention as their importance has been recognized, and as consumers around the world have struggled to balance busy lives with fitness and proper diet. Despite the recognized importance of good health, consumers, on average, are becoming increasingly obese. This has resulted in a strong demand for devices and methods that assist individuals in setting and reaching dietary and fitness goals. Currently available devices and methods fail to meet the needs of average consumers.

There are serious problems with conventional weight loss programs. Weight change is related to the user's net caloric balance, the difference between caloric intake and caloric expenditure. However, determination of caloric intake and caloric expenditure are both problematic.

There are numerous difficulties in accurately determining caloric intake. In some diet programs printed or electronic lists are used that provide the nutrition content of a wide variety of food. The consumer keeps a diet log of all foods consumed each day in order to determine their total nutritional intake. These systems typically are laborious to use and only the most dedicated consumer will accurately use these approaches. For example, a consumer must take the time to accurately record the foods consumed each day. Obviously, recognition of foods consumed is most accurate if done at the time the foods are consumed. However, many individuals feel too rushed to record the foods consumed at the time and postpone recording for later in the day or week. Also, some consumers are embarrassed to be observed recording food intake during or following a meal. This also motivates the user to wait to record their consumption until later. Obviously, accuracy suffers the more time passes between consumption and recordation. An individual may entirely forget that they had a snack or two earlier in the day or week, leading to undercounting of consumption.

Determining total energy expenditure is also difficult. The total energy expenditure of a person comprises a resting metabolic component and a physical activity component. Total energy expenditure (TEE) is the sum of resting energy expenditure (REE) and activity energy expenditure (AEE), i.e. TEE=AEE+REE. Weight loss occurs if total energy expenditure (TEE) exceeds total caloric intake over a given time period. As discussed by Remmereit in U.S. Pat. No. 6,034,132, 70 percent of total energy expenditure for a typical person is due to their resting metabolic rate (RMR). In a conventional diet program, RMR is estimated from the height, weight, age, and gender of the person, for example using the Harris-Benedict equation. This equation, well known to those skilled in the nutritional arts, is given in U.S. Pat. No. 5,839,901 to Karkanen, and in U.S. Pat. No. 5,639,471 to Chait et al. There are serious inadequacies in using the Harris-Benedict equation (or any similar equation) in a weight loss program. The Harris-Benedict equation provides only an estimated RMR, which is an average value for people of similar height, weight, age, and gender. However, due to natural variations in physiology, the equation may not be accurate for a specific individual.

Conventional weight loss programs use an estimated total energy expenditure (TEE) based on estimates of activity levels, and estimates of resting energy expenditure (REE) from the Harris-Benedict equation. However, unless the resting energy expenditure (REE) and the activity energy expenditure (AEE) are estimated accurately, the person's caloric balance cannot be known accurately, and the outcome of a weight loss program is likely to be unsatisfactory.

Some users attempt to track their activity energy expenditure (AEE), either for weight loss or general fitness purposes. In the simplest approach the individual maintains an exercise log of activities conducted, such as distances walked or jogged. Various graphs and tables can then provide an estimate of the calories burned during these activities. As with recording consumption, an individual may fail to accurately record the type and duration of activity undertaken leading to inaccurate recordation. Also, an individual may not know how far or fast they ran or walked. A variety of pedometers are available to assist with this task. Pedometers include some type of stride counter in order to count the number of strides or paces taken by the individual. The devices may be calibrated to allow them to determine the distance traveled with reasonable accuracy. Through the use of timers, they may also be able to determine the speed and duration of activity. Pedometers typically fail to take into consideration changes of elevation, changes in length of stride and changes in intensity. For example, a runner may combine slow walking with brisk running during an exercise session. By combining total number of strides and duration of activity, the pedometer may only determine average speed, not instantaneous speed.

U.S. Pat. Nos. 6,002,982 and 6,148,262 to Fry; U.S. Pat. No. 6,013,007 to Root et al; U.S. Pat. No. 6,009,138 to Slusky; and U.S. Pat. No. 6,032,108 to Seiple et al each disclose improved activity monitors utilizing a global positioning system (GPS) and are hereby incorporated by reference. The devices track an individual's position over time, using the GPS network. By periodically or instantaneously comparing position and time, such a device is capable of determining a performance profile with better accuracy than a typical pedometer.

Devices are also available for monitoring and tracking heart rate. The most popular of these devices are sold by Polar of Finland. These heart rate monitors includes a wristwatch-style display unit and a chest strap with a heart rate sensor. The chest strap and display unit communicate wirelessly. The devices are capable of accurately monitoring heart rate, which correlates reasonably well with exercise intensity. Advanced devices include the ability to track heart rate over time so that a heart rate profile may be produced.

Each of the above-discussed activity monitors fails to consider the dietary intake portion of total health management. Instead, they are directed merely to activity monitoring. In light of this, there remains a need for a device that combines activity monitoring and diet monitoring, that is easy to use and provides accurate results.

U.S. Pat. No. 6,513,532 recognizes the need for devices to help track calorie consumption and activity level but fails to provide a simple means to quickly enter all nutrition data into the device other than by follow-up at a later date once the device is attached to a computer. Bar code scanning or image recognition (of photos taken from the device) are identified as a means to provide “information about consumption” but explains little of how the bar code provides the information on consumption. No data base is provided on the device for this correlation. No external labels are provided for on the food product in addition to bar codes or other data code are provided that enable the user to know that the information exists within the database. Further '532 discloses that at point of consumption such as in a restaurant or in a grocery store the device may be connected to a computer or a scanner with respect to information on consumption. While there is value in getting the information regarding dietary intake in restaurants, connection to computers in restaurants is not likely to be a rational approach. Further, no real time manual entry is afforded by this system to provide support for less than complete records in real time.

U.S. Pat. No. 6,978,221 issued to Rudy describes a dietec scale that enables Universal Product Codes to be read by a bar code reader and weight of the enclosed food to be measured and have FDA Nutrition Facts entered. This patent also describes means to enter information for foods such as meat or vegetables that have no UPC codes or FDA Nutrition Fact labeling and to develop codes for these items that can be entered by hand. By requiring a scale, this system has an awkward feature that reduces its value for portability. By not tracking energy expenditure it is only able to track nutrient and caloric intake—and does nothing to track or enable tracking of caloric expenditure. All automatic input is limited to bar code input and it provides no information to the consumer to differentiate between items that may have additional information in the data base versus those that they are forced to enter into the data base from the limited information on the product label. Further, no translation of the output of these systems into standardized weight loss codes used by leading weight loss firms is provided—such as Weight Watchers and Jenny Craig which each have their own system for talking about dietary intake, weight management, and exercise.

U.S. Pat. No. 6,790,178 issued to Mault et al describes personal digital assistants which provides the controls display, and processing circuitry for a physiological monitor module. It describes the potential to recognize modules using stored codes, bar code scanning, or other approaches. Mault '178 even includes a calorimetry module which provides a good means for measuring current rate at which calories are being consumed as well as to a pedometer. Mault describes a two unit system which has little value as a portable everyday use device. Mault provides no basic means for entering standardized codes into the PDA devices.

U.S. Pat. No. 6,878,885 issued to Miller-Kovach describes a process of controlling weight in which a selection of food servings is based on calculated point value and a range of allotted daily points which is adjusted for weight change. This process can include electronic calculators.

U.S. Pat. No. 6,341,295 issued to Stotler also recognizes the needs to track calorie intake and calorie consumption through physical activity in a portable device and presents a means of doing so with simplified icons.

U.S. Pat. Nos. 5,796,640; 5,704,350; 5,412,560; 4,951,197; 4,891,756; and 4,796,182 also disclose means for managing weight using calculators and computer programs.

In addition to nutritional and caloric information there is a need for consumers to have better information regarding the both medicinal drugs they have consumed and the recreational drugs they have consumed such as alcohol, tobacco, and caffeine.

There is also value in having this information distributed to others. For example groups working on losing weight can support one another emotionally if the information were available on a common website or even emailed to one another. This is central to the business model of Weight Watchers which generated $2.5 billion in revenue in 2004.

In addition medication dosage information would be valuable if it were possible to share and transmit. For physicians compliance to drug regimes is a critical issue in the maintenance of health and management of disease. A portable device that enables an unsophisticated patient to enter data when medications are taken would provide excellent documentation for physicians with respect to medication dosing compliance. The same portable device could be set up so that it provides an alert to the owner that medications should be taken based on the last information entered. For example, when one misses a dosing in a medication at the proper time it would enable the patient to be alerted to when the next dosing should be taken to more rapidly return to the intended dosing regime. Such a device has significant utility in running clinical trials in medication development. Such a device also has significant utility for family members, particularly of the elderly to know they are taking their medications appropriately.

U.S. Pat. No. 6,993,573 disclose a means of retrieving internet content through image recognition of a photographed barcode or image, however it relies on a data base and image recognition system that is not stored on the mobile device, requiring information to be sent out over the internet and retrieved. A poor photograph of the image or bar code would require transmission over the internet, and likely a waiting period before the user would know if the photograph had been made appropriately and whether the information was successfully secured in the photograph or if the information is available at all.

U.S. Pat. No. 6,542,933 discloses a special bar code which is scanned, and the decoded data in parsed into two portions (a server ID and an item ID), with the server ID used to retrieve a URL template that is sent back to the device.

Systems exist in the prior art that allow a user to scan a bar code such as a product UPC code (or other machine-readable indicia), decode the bar code data, and send the decoded bar code data to an offsite server computer, where the server computer looks up a URL associated with that bar code in a database and sends the retrieved URL back to the user's computer. A browser at the user's computer then uses the URL to retrieve web content associated with the URL. This type of system, for example disclosed in U.S. Pat. Nos. 5,978,773 and 6,199,048. This retrieved information does not integrate with longitudinal data obtained over time for the end user.

Today providing portable access to information through bar code scanning and radio frequency identification is a reality. Microvision provides portable bar code scanners such as the Flic Laser Bar Code System which can be used to transmit information to cell phone based computing platforms using blue tooth. Symbol Technologies also provides portable and even wearable mobile computers, together with bar code scanners, and radio frequency identification (RFID) solutions.

The Nutrition Labeling and Education Act of 1990 (NLEA) provides FDA with specific authority to require nutrition labeling of most foods regulated by the Agency. Most products carry a Universal Product Code to enable them to be scanned with a bar code reader (or other imaging device if desired). A database correlating current (and additional) information would have great value if a user could secure it and manage it in a longitudinal fashion.

A pedometer (also known as a Tomish-meter) or step counter is a device, usually portable and electronic, which counts each step a person makes. Used originally by sports and physical fitness enthusiasts, pedometers are now becoming popular as an everyday exercise measurer and motivator. Often worn on the belt and kept on all day, it can record how many steps the wearer has walked that day, and thus the kilometres/miles (Distance=number of steps×step length). Some pedometers will also record movements other than walking, such as bending to tie one's shoes, though the most advanced devices record fewer of these ‘false steps’. Step counters can give encouragement to compete with oneself in getting fit and losing weight. A total of 10,000 steps per day (equivalent of 5 miles or 8 km) is recommended by some to be the benchmark for an active lifestyle, though this point is debated among experts [1]. Step counters are being integrated in an increasing number of portable consumer electronic devices such as music players and mobile phones.

Pedometers can also be a motivation tool for people wanting to increase their physical activity. A study published in The Journal of the American Board of Family Medicine [2] showed people who wore a pedometer and kept a log of their steps, were more likely to continue walking during a 6 month period than those who didn't wear a pedometer. The researchers concluded, “In a family medicine setting, pedometer use is easy to implement and well accepted among sedentary, overweight, and obese patients. Regular use of a pedometer seems to increase physical activity in this population for up to 6 months.”

The technology for a pedometer includes a mechanical sensor and software applications to count steps. Early step counters used a mechanical switch to detect steps together with a simple counter. If one shakes these devices, one hears a lead ball sliding back and forth. Today advanced step counters rely on MEMS inertial sensors and a sophisticated software solution to detect steps. These MEMS sensors have either 1, 2 or 3 axis detection of acceleration. The use of MEMS inertial sensors allows for more accurate detection of steps and fewer false positives. However the software technology to interpret the output of the inertial sensor and “make sense of accurate steps” varies widely. The problem is compounded with the fact that in modern day-to-day life such step-counters are expected to count accurately on the belt, in a handbag, in a back pack, in a back pocket and other locations where users frequently carry their devices.

The accuracy of step counters varies widely from device to device. Most step counting devices today count steps at a walking pace on a flat surface reasonably accurately if the device is placed in its optimal position (usually a belt clip). If the device is placed in a user's pocket or handbag, the accuracy is dramatically reduced. Equally, most step counters today falsely count steps when a user is driving in their car or other habitual motions that the device encounters throughout the day. This error accumulates for users with moderate commutes to work. The accuracy is also dependent on the step length the user enters. Pedometers are accurate to within 5% error.

Apple and Nike offer the NIKE+IPOD SPORTS KIT™ which uses a shoe sensor that communicates with a wireless iPod Nano receiver to transmit workout information such as elapsed time, distance traveled, and calories burned. NTT DoCoMo Fujitsu Pedometer Phone is the first integrated phone with a pedometer that works 24 by 7 and counts step like an Omron pedometer. The sensor is made by ADI. This handset was introduced in Japan in 2004 and has sold over 3 million units. The Nokia 5500 Sports Phone uses an embedded 3 axis MEMS inertial sensor to detect the steps a user takes. The pedometer application tracks steps taken, time elapsed and distance traveled. The Sony Ericcsson w170 and W580 WALKMAN″ phones use embedded 2 axis MEMS inertial sensors to detect the steps a user takes. The W710 is a clamshell phone and displays the user's steps on the external display. The W710 must be closed in order for it to count steps. When the step counter is activated, it counts detected steps during the day, and at midnight it stores the counter in a day-by-day history and resets it to zero.

### SUMMARY OF THE INVENTION

The simplest example of system under this invention includes a bar code scanner that the user carries on the waist. The device tracks motion similar to a standard pedometer. The device scanner function has the ability to read in UPC codes for recording consumption of body intake, provides only an auditory confirmation that the data has been scanned, as well as a time stamp for each scan. Upon connecting the device to a computer it connects to a software package that takes the bar code and activity data and associated time codes and after quality check of the data for repeat entries, and likely inappropriate entries, stores it in a longitudinal data base. The longitudinal data base for a given day is tied into database from previous days.

The present invention in its preferred embodiment is a personal digital assistant, such as a cellular telephone, having software for tracking longitudinal data specific to the user, means to retrieve coded data using embedded camera, barcode readers, or RFID scanners, correlating the coded data to a database of such codes maintained on the device, providing means to track and display numerically and graphically the information corresponding to the coded data, and the means to share this information with others, such as over the internet. The personal digital assistant may incorporate alphanumeric data entry and automated entry such as motion or activity sensors. Further in some instances, retrieval of internet based information as described in the prior art described herein, hereby incorporated by reference, may also be appropriate.

An electronic mobile telephone device is disclosed that enables the user to obtain information through a built in bar code scanner or electromagnetic radiation receiver and integrate this information with other information to manage health and disease. Further, methods of using such devices to manage and improve health are provided including management of nutrition, management of caloric intake, establishing dietary recommendations to meet fitness goals, labeling of products where the nutritional data has been entered into the referenced data base, providing information on user activity to other parties, enabling the devices act as keys to other devices,

Further, a pedometer with blue tooth that can communicate with a device capable of storage, presentation, and sharing with other parties enables management of longitudinal activity data that has value for social networking, real time feedback on caloric expenditure, and the means to separate the motion activity from a potentially larger device less suited to tracking physical activity due to size, shape, or the difficulty of incorporating hardware based activity data capture into available electronic systems that may be easily coupled to a blue tooth pedometer.

Further a method of labeling objects with a second label to note that they are entered into the available data base that may or may not have quality control performed on the associated database informational content.

### BRIEF DESCRIPTION OF THE DRAWINGS

FIG. 1 shows a cell phone with means of obtaining information from a bar code scanner and software with related databases.

FIG. 2 is a food label which includes nutritional facts, a UPC symbol, and a coding simplified external labeling to assess the key parameters of the data base visually without scanning.

FIG. 3 shows the critical energy balance for weight loss.

FIG. 4 shows information based on a daily activity monitor and consumption information available to one or more other predefined individuals over the internet.

FIG. 5 shows the daily energy balance of two group members showing their activity, dietary intake, and weight goals.

### DETAILED DESCRIPTION OF THE INVENTIONS

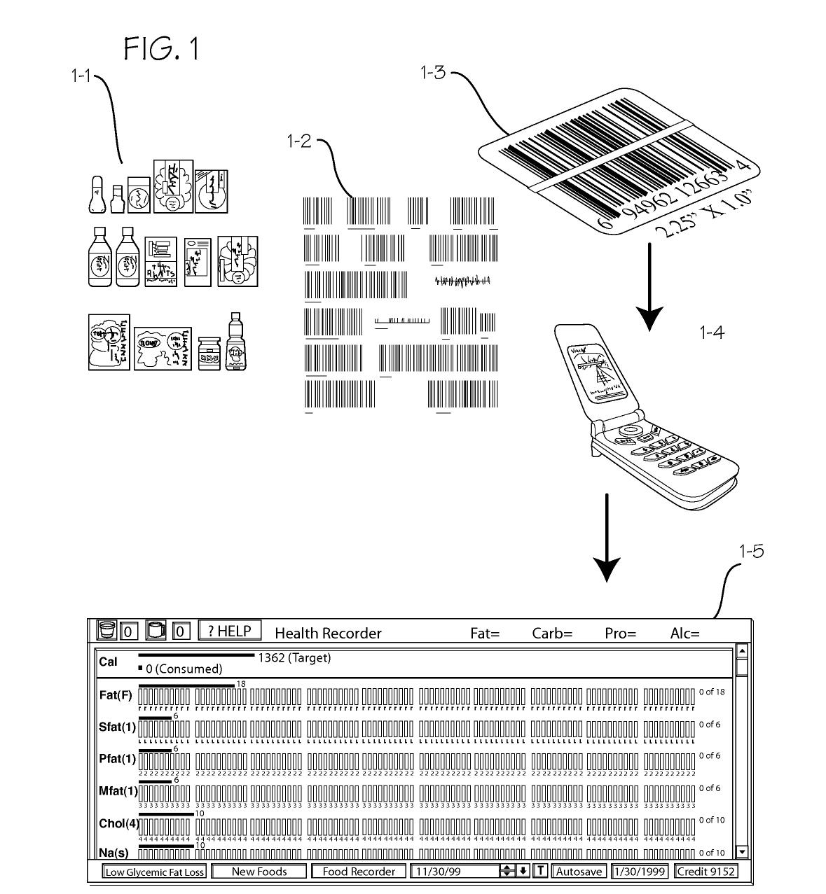

FIG. 1 shows food products, medicines, and alcohols 1-1 are labeled with barcodes 1-2 which are tied to key information for the end user such as nutritional value and the presence or absence of allergens. A scanning device 1-3 is used to capture the information which is stored in an onboard electronic device 1-4. The information is encoded in bar codes or other printed indicia, and placed on various groceries by manufacturers, distributors or purchasers. The printed indicia may include any nutritional information that may be of interest to consumers.

In the preferred embodiment the electronic device is a smart cell phone with database software systems for diet & exercise management and the means to present data in real time graphically in display 1-5. Other smart phones and personal digital systems are also incorporated herein. Alternatively a cell phone may be connected to a commercially available scanner wirelessly using blue tooth, or the electronic device could be used without cell phone capabilities to connect to a computer elsewhere for downloading of data, presentation of results, and sharing of results.

This system enables one to point at bar codes and quickly obtain the information associated with that bar code stored in a longitudinal data base. The system may also enable graphical output of the entered information in a longitudinal fashion.

Cell phone electronic device 1-4 includes a bar code reader, a database, and a data entry module of nutritional information which corresponds to that in food products and medicines, and a healthcare or dietary software module that enables data entry, display, and management. The bar code reader may comprise a convention bar code reader or a built-in cell phone camera along with processing software. In a preferred embodiment of the invention, the cell phone scans the UPC symbol of a food package and the nutritional information of a single portion are displayed on the screen. This may be coupled with information based on whether the food is considered healthy or unhealthy as well as the impact that the consumption of said food will have on daily or weekly balances. In routine usage, the individual scans the carton of half gallon of orange juice in the morning, scans the label on the container of English muffins, and possibly hand enters the data on the grapefruit that is also consumed. At lunch, the individual goes out to lunch and orders a meal at a restaurant and enters data manually—as today restaurants do not yet have information on nutritional content that is readily readable. In the evening the individual has a pre-prepared food (such as lean cuisine, Jenny Craig, or Weight Watchers) in an individual portion with a glass of milk and scans these as well. Thus through the course of the day, nutritional content can be entered such as through Diet & Exercise Assistant for PALM OS® Diet & Exercise Assistant for POCKET PC®, or Diet & Exercise Assistant for WINDOWS PC®. However it may also be more rapidly entered through the bar code reading of the UPC Symbol. Exercise and activity can also be entered manually in this system. Through the use of this system, the time consuming effort of entering data manually is significantly reduced. Where bar codes exist for food products in the data base, there is little effort involved. Should a restaurant have bar codes available for their meals that have been quality checked (an aspect of this invention), the customer would merely scan the label next to the menu item and not need to enter in any manual data whatsoever. Thus, the printed indicia may be provided to users through various media.

An identical system using a cell phone with a RFID code reader, and a data base of information which corresponds to that in food products, and a dietary software module that enables data entry, display, and management is also incorporated herein by reference. It would be used identically to the devices and systems using barcodes, except that it reads RFID tags instead of bar codes.

Using the systems of FIG. 1, and software that estimates caloric targets with manual weight entry, one can track nutritional intake as a function of time. This feedback on the control system that includes manual weight entry provides a means to have a feedback loop to assess the measurements made. Such an approach gives the user a good measure of what they have consumed each day and is valuable because it helps one understand the benefits and costs of a particular course.

In the preferred embodiment, the electronic scanning device of FIG. 1 also includes an accelerometer or pedometer. This enables daily activity to be tracked over time and algorithms of weight management such as calorie target goals to be set based on activity. These may be used in conjunction with manual inputs of exercise activity that will not be apparent on the device such as bicycling, weight lifting, and heart rate. This system provides better closed loop control while also helps minimize the amount of unnecessary data entry and manipulation. The additional feature also enhances health by providing motivation for exercise and activity in a simple fashion.

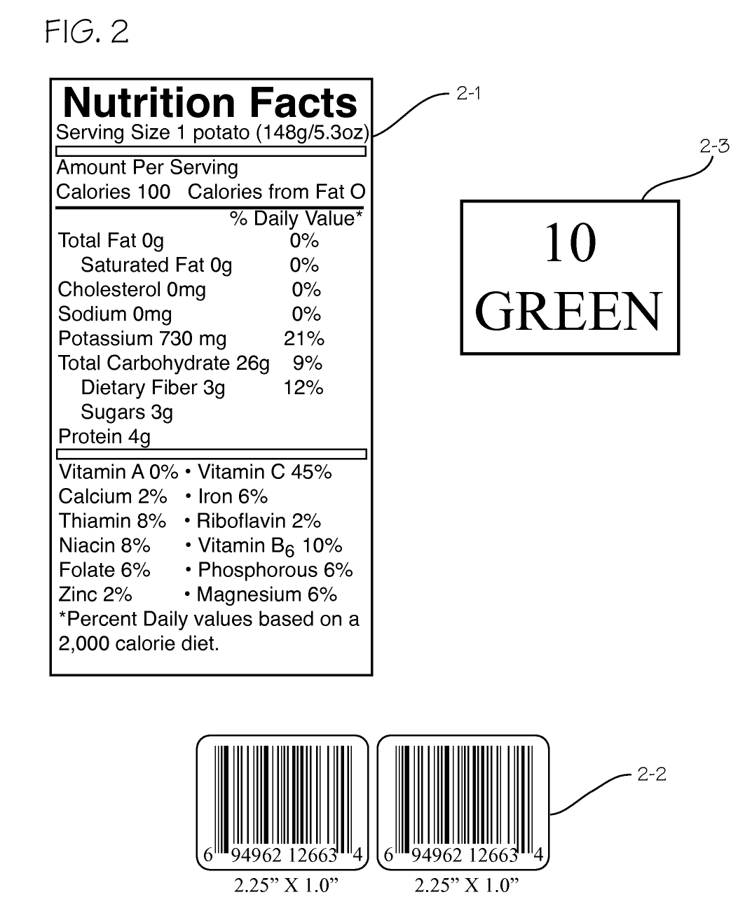

FIG. 2 shows a label on a food product showing that its nutritional information as required by law in label 2-1 and its UPC symbol 2-2, which enable the development of a database that resides on the device. In this fashion real time decisions with knowledge of caloric, fat, salt, or other recommended daily allowances can be made—but they can also be stored into a personal longitudinal data base that enables tracking of these inputs for health. The database may include more information than that typically contained in the nutritional labeling as the database is developed and advanced. An organization that certifies food products, medicines, alcohol, and the like may also incorporate a short hand code, in a form illustrated in label 2-3, to provide the user with either a point value associated with the object or a classification scheme such as a color for certain nutritional regimens. The service that provides these labels and establishes appropriate UPC codes for use with such devices is an aspect of the invention disclosed here. Such coding can be for food and drug.

Clearly products designed to be used with such a system for input into a personal longitudinal data system will make these systems even easier to use, and are also part of this invention. The difficulty of adding up the nutritional content of a home made salad with lettuce, shrimp, avocado, dressing, anchovies, and capers and entering it into the system is nontrivial, and completely eliminated by a personal portion such as we find in standardized food packs such as Weight Watchers, Lean Cuisine, Jenny Craig meals—or even a Big Mac Hamburger at McDonalds. Weight Watchers have produced their own line of cuisine which may be purchased independently at most major grocery chains which merely has a points value that fits into their system, through the application of the methods and devices disclosed here an individual will be able to track not just high level point values but all nutritional information and in real time have an idea of the status as to where they are at on their energy balance. The value of having such food products made and optimized with UPC symbols or RFID coding to enable rapid input of the relevant information is of great value in enabling accurate tracking of the inputs to the system. It may even present a compelling reason for a customer to attend one restaurant which provides such codes on a menu versus one which does not.

Data related to alcohol content on a beverage can be used to estimate the blood alcohol concentration of an individual over time. This real time calculations to provide a better guidance to an individual to know when he or she has a blood alcohol content that is less than safe levels.

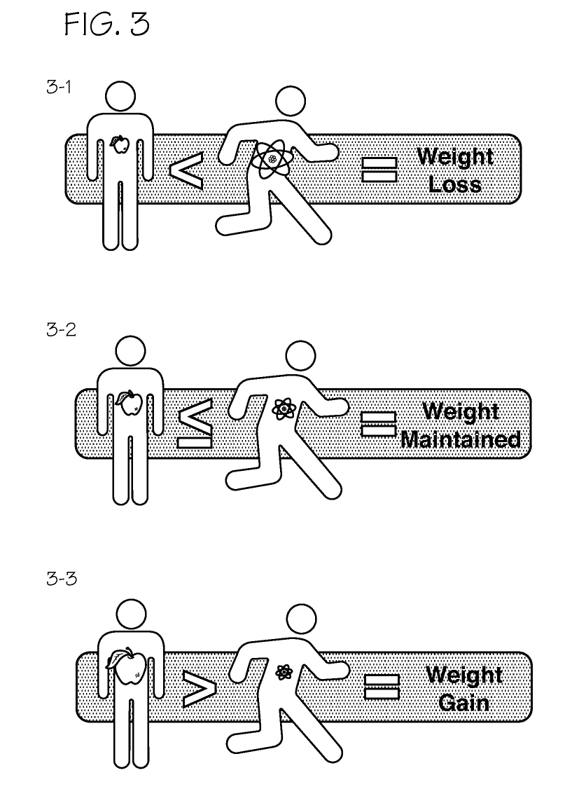

FIG. 3 shows the key issues for energy balance. Where energy expenditure is greater than energy intake a green zone 3-1 can be reached which will decrease weight loss. An orange zone is also possible where there is energy balance with no weight gain or loss 3-2. A red zone where weight increases 3-3 is the red zone and is to be avoided. This simple system enables color coding or other simple metric for the user so that they will always know where they are at during the course of the day. This is highly motivational.

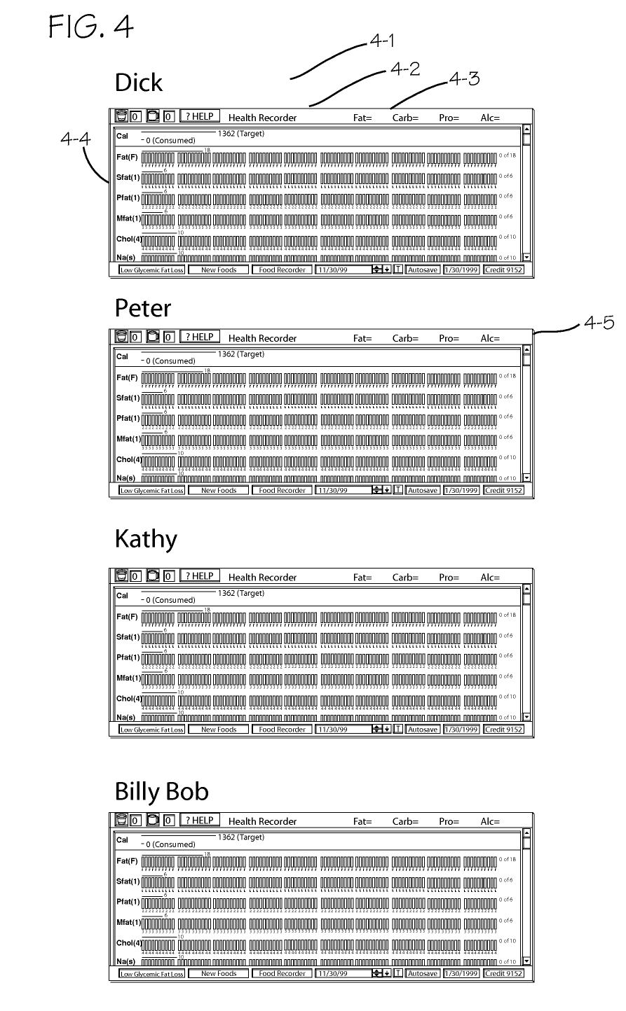

FIG. 4 shows information based on the daily activity monitor and dietary monitor available to a group of predefined friends, family, or other individuals over the internet. Here, the data available to the user can be menu driven and associated with looking at particular metrics such as fat consumption or exercise output such as for Smart Brain (not shown) and can be viewed longitudinally over the course of days, weeks, and months. Information associated with weight, activity, food caloric and nutritional consumption, alcohol consumption, tobacco consumption, and medication consumption can be shared in whole or in part with a number of individuals.

In the preferred example the user is primarily interested in fitness and shares information with others as a means of holding himself accountable for actions and receiving input and support from others. With wireless updates such as are possible from cell phone, realtime activity from an onboard pedometer or motion sensor can be seen and entered food consumption tracked. A real time system with updates would literally enable friends to see the activity in real time providing significant motivation to get active. Such a system would enable the same group function of a Weight Watchers meeting, but could enable it in real time on an hour by hour basis. It could also enable it without regard to physical location as it could be used among siblings who live in different states or even different countries. The system thus provides for tracking and sharing exercise activity among a social network and includes a data sharing system that enables the longitudinal activity data to be shared with other members of the social network. This includes a means to enter longitudinal data regarding caloric or nutritional intake.

Such a system could also be very valuable for physicians or family members who value tracking data for medication compliance. The act of scanning the medication package at the time of taking the medication enters the dosage into a system that can be transferred wirelessly to the internet site to enable the information to be available over the internet. Further the correlation of medication to outcomes in clinical trials to patient compliance of medications would also be enabled for clinical studies. Also, the user may manually enter other parameters such as blood sugar levels, blood pressure, etc., gained from measurements taken by the user during the course of a day, and this can be displayed in a comparative display to illustrate the fluctuation of blood sugar or blood pressure with food intake, drug intake, and exercise levels.

FIG. 4 shows activity of four colleagues showing their activity, dietary intake, and weight goals. Here user 4-5 has a gold border denoting a leading position with respect to a predefined agreed upon metric such as steps walked, activity, fewest calories consumed, most vitamin A, and almost any other metric one can imagine. For individuals who wish to cut back on drinking alcohol, this approach could serve to track the number of drinks one has had as a real time disincentive to fall off the wagon due to the group support aspects.

The value proposition of being able to (i) obtain the data in real time in a simple fashion and (ii) transfer the data to a longitudinal data base, and (iii) share this data with others enabled by the user are numerous. In the preferred embodiment of this invention, the usage of the system of this invention is to monitor and maintain personal wellness. This applications includes group weigh loss, exercise and performance training, and medication management which can all benefit from peer, physician, and trainer (here called partner users) interaction and involvement with the data interaction.

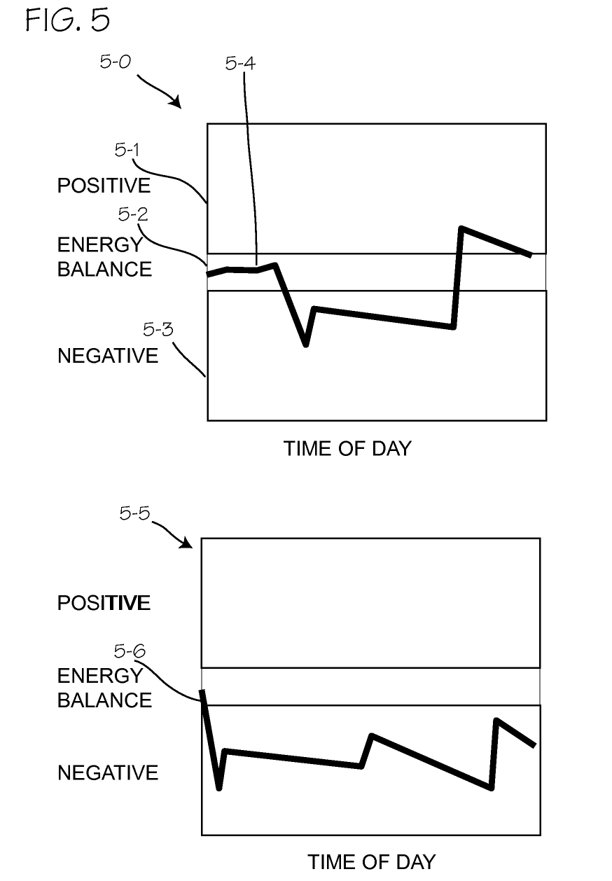

FIG. 5 shows integrated data available on the website showing two users 5-0 and 5-5 of such a system and method for tracking energy intake and consumption. Three zones are provided where energy expenditure is less than intake at zone 5-1, where energy balance is approximately achieved at zone 5-2, and where energy expenditure is greater than energy intake in zone 5-3. Here, time course 5-4 for user 5-0 shows that that modest intake of energy over time that exceeded output was followed by significant output such as by exercise. This was then followed by a sharp rise in the middle of the day likely due to lunch and a slow decrease through the afternoon due to more modest activity. Although active, the individuals evening has a significant spike due to intake resulting in less than positive results. This enables the user to not only alter behavior in a very clear way, but also helps to note when data entry has or has not been appropriately introduced—as the absence of a spike at a meal time suggests no data entry was performed. User 5-5 has a very different energy consumption process and is able to stay in the green or weight loss zone throughout the course of the day. In fact, the evening energy intake can be specified to stay in the green zone by the user.

Broadly speaking, the method described above, and the system upon which it is implemented, provide for the accomplish of a method for obtaining and tracking longitudinal data for the maintenance and management of health of an individual according the steps of (1) establishing a standard for placing or incorporating indicia regarding nutritional content of in or on numerous groceries; (2) placing such indicia on numerous groceries; (3) providing a plurality of users with electronic means for reading said indicia and storing information regarding the nutritional content of numerous groceries in a database; (4) operating said means for reading and storing information to obtain nutritional information for a substantial portion of groceries consumed by the respective users; and (5) displaying in a graphical display cumulative nutritional intake for a period of time. The same method is implemented on corresponding systems for tracking exercise and energy expenditure for users, and tracking the pharmaceutical content of any drugs consumed by the user, by providing a means for detecting physical activity of the user during the period of time and correlating that physical activity to an estimate of energy expenditure by the user and storing said information regarding energy expenditure in a database calculating the calorie content of cumulative nutritional intake for the period of time, and/or establishing a standard for placing or incorporating indicia regarding pharmaceutical content of numerous drugs, placing the indicia on drugs or their containers; providing a plurality of users with electronic means for reading said indicia and storing information regarding the pharmaceutical content of numerous drugs in a database and operating said means for reading and storing information to obtain pharmaceutical content information for a substantial portion of drugs consumed by the users. All of the information can be displayed graphically, as described above, to assist the users or others associated with their care in tracking food and drug consumption and exercise.

A pedometer for use with the system may use a blue tooth or other wireless data transmission capability that is coupled to a personal device which in turn has the ability to update broader networks in a timely fashion. In usage the pedometer transmits the data on the wearers activity to the remote device such as a cell phone upon which the data may be stored and graphically presented. If the remote device also incorporates even the simplest data entry system for tracking caloric and nutritional consumption, potentially including internal accounting of metabolic caloric and nutritional consumption over time (which could be calibrated to the wearer), the user would then have a closed loop system that would be able to identify whether one was a head or behind a particular objective. In turn, the user may elect to have this information shared on a timely fashion through the internet to other users in a social network. The ability to share this data over networks has enormous value in providing feedback to personal wellness. This embodiment of this invention has significant value in that it can be created in a very cost effective fashion and need not require any modifications to a remote device. The same external pedometer could be used with cell phones by a number of manufacturers with only software modifications. Further, the remote device may initially not be a device with varied software platforms, but may be a standardized desktop computer platform that avoids the requirement of creating a family of software products appropriate for all cell phone or personal digital assistant software platforms.

Additional preferred embodiments include (1) the means to perform remote data entry to add information that is not possible to enter through the scanning function, (2) to remotely transmit this data to either retrieve additional data or enable real time sharing with partner users, and/or (3) and the ability to have quick keys that can be entered such as when a standard meal is consumed or a standard workout regime is completed.

These systems can be configured to be compatible with existing dietary programs such as Weight Watchers, Jenny Craig and the like and to use their nomenclature of dietary values.

The system can also be incorporated into social or professional networking internet sites to create a means to enable friends or associates to be involved in ones support group.

I claim:

1. A method for obtaining and tracking longitudinal data for the maintenance and management of health of a user on a personal digital assistant comprising the steps of:
a. capturing a coded data identifier,
b. processing, on the personal digital assistant the captured coded data identifier to identify related informational content from a data base stored on the personal digital assistant,
c. entering related informational content into a longitudinal database stored on the personal digital assistant, and
d. viewing the compiled longitudinal data from the personal digital assistant; and
e. sharing the compiled longitudinal data of the personal digital assistant on a social network with other members of the social network.

2. The method of claim 1 further comprising using a bar code symbol as the coded data identifier.

3. A method for obtaining and tracking longitudinal data for the maintenance and management of health of an individual, said method comprising:
establishing a standard for placing or incorporating indicia regarding content on numerous packages containing food or medicine;
placing such indicia on numerous packages containing food or medicine;
providing a plurality of users with electronic means for reading said indicia and storing information regarding the content of numerous packages containing food or medicine in a database;
operating means for reading and storing information to obtain information for packages containing food or medicine consumed by the respective users;
displaying in a graphical display the information for packages containing food or medicine consumed by the respective users for a period of time; and
sharing the stored information on a social network with other members of the social network.

4. The method of claim 3 further comprising:
providing a means for detecting physical activity of the user during the period of time and correlating that physical activity to an estimate of energy expenditure by the user and storing said information regarding energy expenditure in a database;
calculating the calorie content of food consumed by the respective users for a period of time;
displaying in a graphical display a comparison of the calorie content and the energy expenditure for the period of time; and
sharing the graphical display a comparison of the calorie content and the energy expenditure on a social network with other members of the social network.

5. The method of claim 3 further comprising:
establishing a standard for placing or incorporating indicia regarding pharmaceutical content of numerous drugs;
placing such indicia on numerous drugs;
providing a plurality of users with electronic means for reading said indicia and storing information regarding the pharmaceutical content of numerous drugs in a database;
operating said means for reading and storing information to obtain pharmaceutical content information for drugs consumed by the respective users;
displaying in a graphical display the pharmaceutical content information for drugs consumed by the respective users on a social network with other members of the social network.

6. The method of claim 4 further comprising:
displaying in a graphical display a comparison of cumulative nutritional intake and nutritional intake for a period of time.

7. The method of claim 5 where said drug is selected from the set of alcohol, caffeine, and nicotine.

8. The method of claim 1 further comprising capturing a coded data identifier from a restaurant menu with a coded data identifier associated with a menu item.

9. The method of claim 3 further comprising:
establishing a standard for placing or incorporating indicia regarding nutritional content of restaurant items;
placing such indicia on restaurant menus;
providing a plurality of users with electronic means for reading said indicia and storing information regarding the restaurant items in a database;
operating said means for reading and storing information to obtain regarding the restaurant items for a substantial portion of restaurant items consumed by the respective users;
displaying in a graphical display regarding the restaurant items for a substantial portion of regarding the restaurant items consumed by the respective users on a social network with other members of the social network.
